(19) United States
(12) Patent Application Publication
(10) Pub. No.: US 2021/0145349 A1
(43) Pub. Date: May 20, 2021

(54) HEAD-MOUNTED SPECTRUM SENSING DEVICE

(71) Applicant: HON HAI PRECISION INDUSTRY CO., LTD., New Taipei (TW)

(72) Inventors: I-THUN LIN, Tu-Cheng (TW); HSIN-SHUN HUANG, Tu-Cheng (TW); YING-JHIH HONG, Tu-Cheng (TW)

(21) Appl. No.: 16/851,307

(22) Filed: Apr. 17, 2020

(30) Foreign Application Priority Data
Nov. 15, 2019 (TW) 108141605

(51) Int. Cl. A61B 5/00; G01R 33/26; G01J 5/00; A61B 5/01; A61B 5/02; A61B 5/05; A61B 8/08

(52) U.S. Cl. CPC A61B 5/4088; A61B 2562/0223; G01J 5/0025; A61B 5/015; A61B 5/02028; A61B 5/05; A61B 5/02007; A61B 5/0042; A61B 5/7275; A61B 5/7257; A61B 5/6803; A61B 5/0035; A61B 8/0808; A61B 8/5261; A61B 8/5223; G01J 2005/0077; G01J 2005/0085; A61B 2562/0204; G01R 33/26

(57) ABSTRACT

A head-mounted spectrum sensing device is provided which includes an acoustic system, a magnetic system, an ultrasound system, and a control and processing system. The head-mounted spectrum sensing device can judge a risk of dementia according to a thermal image with blood velocity information, a thermal image with magnetic flux information, and a thermal image with ultrasound information.

### FIELD

The present disclosure relates to medical equipment technical field, and more particularly to head-mounted medical equipment.

### BACKGROUND

Dementia is a broad category of brain diseases that cause a long-term and often gradual decrease in the ability to think and remember that is great enough to affect a person's daily functioning. Recently, more and more people suffer a dementia. However, after the patient has been found himself suffer a dementia it can be too late to receive a treatment.

What is needed, therefore, is fast detection equipment which can detect the risk of dementia at an early stage.

### BRIEF DESCRIPTION OF THE DRAWINGS

Many aspects of the embodiments can be better understood with reference to the following drawings. The components in the drawings are not necessarily drawn to scale, the emphasis instead being placed upon clearly illustrating the principles of the embodiments. Moreover, in the drawings, like reference numerals designate corresponding parts throughout the several views.

FIG. 1 is a schematic view of one embodiment of a head-mounted spectrum sensing device.

FIG. 2 is a schematic view of the acoustic system of the head-mounted spectrum sensing device.

FIG. 3 is a schematic view of the magnetic system of the head-mounted spectrum sensing device.

FIG. 4 is a schematic view of the ultrasound system of the head-mounted spectrum sensing device.

FIG. 5 is a schematic view of the control and processing system of the head-mounted spectrum sensing device.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. The drawings are not necessarily to scale, and the proportions of certain parts may be exaggerated to better illustrate details and features. The description is not to be considered as limiting the scope of the embodiments described herein.

Several definitions that apply throughout this disclosure will now be presented.

The connection can be such that the objects are permanently connected or releasably connected. The term “outside” refers to a region that is beyond the outermost confines of a physical object. The term “inside” indicates that at least a portion of a region is partially contained within a boundary formed by the object. The term “substantially” is defined to essentially conforming to the particular dimension, shape or another word that substantially modifies, such that the component need not be exact. For example, substantially cylindrical means that the object resembles a cylinder, but can have one or more deviations from a true cylinder. The term “comprising” means “including, but not necessarily limited to”; it specifically indicates open-ended inclusion or membership in a so-described combination, group, series and the like.

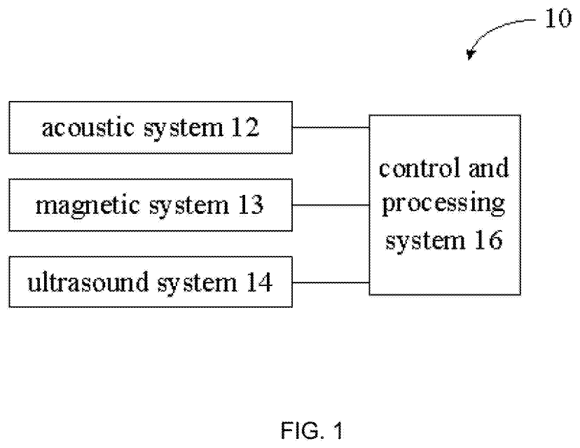

FIG. 1 shows an embodiment of a head-mounted spectrum sensing device 10. The head-mounted spectrum sensing device 10 includes an acoustic system 12, a magnetic system 13, an ultrasound system 14, and a control and processing system 16. The control and processing system 16 is configured to receive signals collected by the acoustic system 12, the magnetic system 13, and the ultrasound system 14. The control and processing system 16 is also configured to control the acoustic system 12, the magnetic system 13, and the ultrasound system 14.

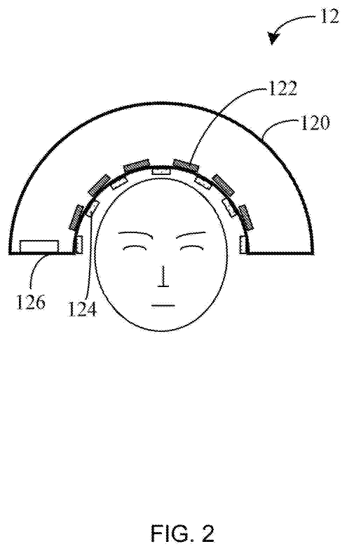

Referring to FIG. 2, the acoustic system 12 includes a first shell 120, a surface acoustic sensing unit 122, a first thermal sensing unit 124, and a first fusion unit 126. The acoustic system 12 is configured to obtain blood velocity and a first thermal image, and then mark the blood velocity at a corresponding position in the first thermal image to generate a thermal image with blood velocity information.

The first shell 120 is configured to support the surface acoustic sensing unit 122, the first thermal sensing unit 124, and the first fusion unit 126. The first shell 120 can be designed to a helmet-like shape, such as a hemispherical shape, so that the user can wear it on the head in the detecting process.

Defending an enclosed space surrounded by each surface as the inside of the first shell 120, and defending the space outside the enclosed space as the outside of the first shell 120. The surface acoustic sensing unit 122, the first thermal sensing unit 124, and the first fusion unit 126 can be located in the inside of the first shell 120, or located in the outside of the first shell 120, more specifically, on the outer surface near the users' head.

In one embodiment, the surface acoustic sensing unit 122 is located in the inside of the first shell 120, more specifically, on the inner surface near the users' head. And the first thermal sensing unit 124 is located in the outside of the first shell 120, more specifically, on the outer surface near the users' head.

The surface acoustic sensing unit 122 is configured to obtain blood velocity, and then predict whether there is a vascular occlusion. In one embodiment, the surface acoustic sensing unit 122 includes a plurality of flexible surface acoustic wave sensors (SAW sensor). The SAW sensor can be made of a zinc oxide piezoelectric film on a polyimide substrate.

The first thermal sensing unit 124 is configured to obtain temperature distribution of the brain and generate a first thermal image according to the temperature distribution. In addition, the temperature distribution can also be used to predict whether there is inflammation in the brain.

The first fusion unit 126 marks the blood velocity at a corresponding position in the first thermal image to generate a thermal image with blood velocity information. The first fusion unit 126 can be a microprocessor.

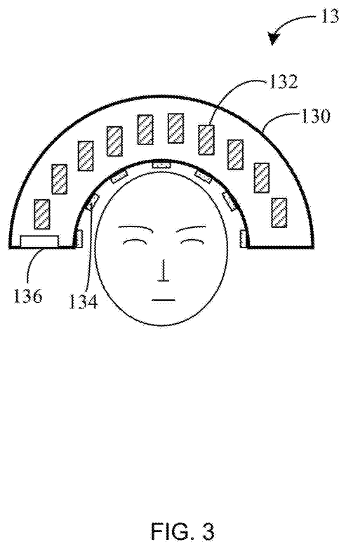

Referring to FIG. 3, the magnetic system 13 includes a second shell 130, a magnetic sensing unit 132, a second thermal sensing unit 134, and a second fusion unit 136. The magnetic system 13 is configured to obtain magnetic flux and a second thermal image, and then mark the magnetic flux at a corresponding position in the second thermal image to generate a thermal image with magnetic flux information.

The second shell 130 is configured to support the magnetic sensing unit 132, the second thermal sensing unit 134, and the second fusion unit 136. The second shell 130 can be designed to a helmet-like shape, such as a hemispherical shape, so that the user can wear it on the head in the detecting process.

Defending an enclosed space surrounded by each surface as the inside of the second shell 130, and defending the space outside the enclosed space as the outside of the second shell 130. The magnetic sensing unit 132, the second thermal sensing unit 134, and the second fusion unit 136 can be located in the inside of the second shell 130, or located in the outside of the second shell 130, more specifically, on the outer surface near the users' head.

In one embodiment, the magnetic sensing unit 132 is located in the inside of the second shell 130, more specifically, on the inner surface near the users' head. And the second thermal sensing unit 134 is located in the outside of the second shell 130, more specifically, on the outer surface near the users' head.

The magnetic sensing unit 132 is configured to obtain magnetic flux, and then predict neurological changes in the brain. In one embodiment, the magnetic sensing unit 132 includes a high-sensitivity magnetic flux sensor based on

Nitrogen-vacancy Diamond to measure brain magnetic signals below nT. The Nitrogen-vacancy Diamond sensor can effectively reduce the intensity of the uniform magnetic field applied to the outside (less than 1 Tesla), and forms a miniaturized monitoring system.

The second thermal sensing unit 134 is configured to obtain temperature distribution of the brain and generate a first thermal image according to the temperature distribution. In addition, the temperature distribution can also be used to predict whether there is inflammation in the brain.

The second fusion unit 136 marks the magnetic flux at a corresponding position in the second thermal image to generate a thermal image with magnetic flux information. The second fusion unit 136 can be a microprocessor.

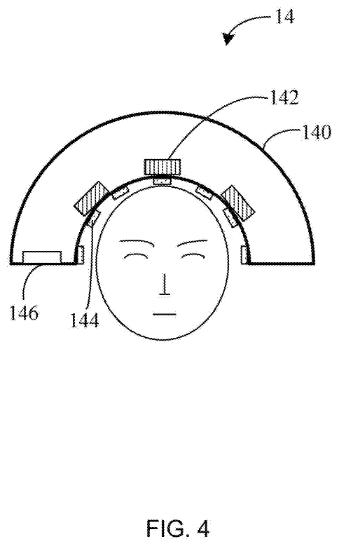

Referring to FIG. 4, the ultrasound system 14 includes a third shell 140, an ultrasound sensing unit 142, a third thermal sensing unit 144, and a third fusion unit 146. The ultrasound system 14 is configured to obtain ultrasound echoes and a third thermal image, and then mark the ultrasound echoes at a corresponding position in the second thermal image to generate a thermal image with ultrasound information.

The third shell 140 is configured to support the ultrasound sensing unit 142, the third thermal sensing unit 144, and the third fusion unit 146. The third shell 140 can be designed to a helmet-like shape, such as a hemispherical shape, so that the user can wear it on the head in the detecting process.

Defending an enclosed space surrounded by each surface as the inside of the third shell 140, and defending the space outside the enclosed space as the outside of the third shell 140. The ultrasound sensing unit 142, the third thermal sensing unit 144, and the third fusion unit 146 can be located in the inside of the third shell 140, or located in the outside of the third shell 140, more specifically, on the outer surface near the users' head.

In one embodiment, the ultrasound sensing unit 142 is located in the inside of the third shell 140, more specifically, on the inner surface near the users' head. And the third thermal sensing unit 144 is located in the outside of the third shell 140, more specifically, on the outer surface near the users' head.

The ultrasound sensing unit 142 is configured to obtain ultrasound echoes. In one embodiment, the ultrasound sensing unit 142 includes an ultrasonic signal transmitting module and an ultrasonic signal receiving module. The received ultrasound echoes can be further transformed into an image via Fourier transform.

The third thermal sensing unit 144 is configured to obtain temperature distribution of the brain and generate a first thermal image according to the temperature distribution. In addition, the temperature distribution can also be used to predict whether there is inflammation in the brain.

The third fusion unit 146 marks the ultrasound echoes at a corresponding position in the third thermal image to generate a thermal image with ultrasound information. The third fusion unit 146 can be a microprocessor.

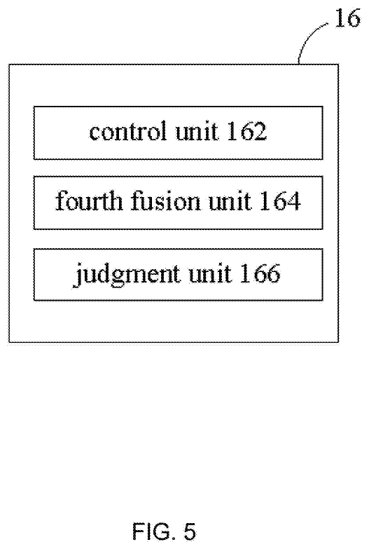

Referring to FIG. 5, the control and processing system 16 includes a control unit 162, a fourth fusion unit 164, and a judgment unit 166. The control and processing system 16 is configured to receive brain spectrum information obtained by the acoustic system 12, the magnetic system 13, and the ultrasound system 14. The brain spectrum information includes the thermal image with blood velocity information which is obtained by the acoustic system 12, the thermal image with magnetic flux information which is obtained by the magnetic system 13, and the thermal image with ultrasound information which is obtained by the ultrasound system 14. The control and processing system 16 is further used to analyze and integrate the brain spectrum information to obtain spectrum fusion information, and to determine whether the user is at risk of dementia according to the spectrum fusion information.

The control and processing system 16 is also configured to control the operation of the above units.

In one embodiment, the acoustic system 12 transmits the thermal image with blood velocity information to the control and processing system 16 by wire or wirelessly. Similarly, the magnetic system 13 transmits the thermal image with magnetic flux information to the control and processing system 16 by wire or wirelessly, and the ultrasound system 14 transmits the thermal image with ultrasound information to the control and processing system 16 by wire or wirelessly.

The fourth fusion unit 164 analyzes and integrates the thermal image with blood velocity information, the thermal image with magnetic flux information, and the thermal image with ultrasound information to obtain the spectrum fusion information.

In one embodiment, the fourth fusion unit 164 fuses blood velocity (denoted as v), magnetic flux (denoted as m), and ultrasound echoes (denoted as s) to form the spectrum fusion information. The spectrum fusion information includes both acoustic, magnetic, and ultrasound information. The spectrum fusion information at a certain position can be expressed as A (x, y, z, v, m, s), where x, y, z are the position coordinates, v is the blood velocity obtained by the acoustic system 12, m is the magnetic flux obtained by the magnetic system 13, and s is the ultrasound echoes obtained by the ultrasound system 14.

The judgment unit 166 is configured to receive the spectrum fusion information sent by the fourth fusion unit 164 and determine whether the user has the risk of dementia. The judgment unit 166 can be connected to the third fusion unit 146.

Specifically, the judgment unit 166 compares the spectrum fusion information (A1, A2, A3, . . . ) with a big data database. A1, A2, A3, . . . are spectrum fusion information collected from a user at different times. ΔA1, ΔA2, ΔA3, . . . are changes between A1, A2, A3, . . . and the big data database, respectively. The judgment unit 166 predicts the probability of disease occurrence based on ΔA1, ΔA2, ΔA3, . . . and outputs a risk alert to the user.

In one embodiment, the H1 is defined as a first changes threshold, H2 is defined as a second changes threshold, and H1<H2. When the changes of the spectrum fusion information (ΔA1, ΔA2, ΔA3, . . . ) is less than the first changes threshold H1, the judgment is “no changes”. When the changes of the spectrum fusion information (ΔA1, ΔA2, ΔA3, . . . ) is greater than or equal to the first changes threshold H1 and less than or equal to the second changes threshold H2, the judgment is “slight changes”. When the changes of the spectrum fusion information (ΔA1, ΔA2, ΔA3, . . . ) is greater than the second changes threshold H2, the judgment is “obvious changes.” The first changes threshold H1 and the second changes threshold H2 can be set according to the actual situation. The changes of the spectrum fusion information (ΔA1, ΔA2, ΔA3, . . . ) can be obtained by using Convolutional Neural Network (CNN), Recurrent Neural Network (RNN), and/or Deep Neural Network (DNN).

The control and processing system 16 can include user interface so that the user can operate the head-mounted spectrum sensing device 10. The control and processing system 16 can also be connected to the mobile electronic device of the user, such as mobile phone, by wires or wireless. Thus, the user can operate the head-mounted spectrum sensing device 10 by downloading an APP. In operation of the head-mounted spectrum sensing device 10, the acoustic system 12, the magnetic system 13, and the ultrasound system 14 are worn on the head of the user.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms used in the claims.

Depending on the embodiment, certain of the steps of methods described may be removed, others may be added, and the sequence of steps may be altered. The description and the claims drawn to a method may include some indication in reference to certain steps. However, the indication used is only to be viewed for identification purposes and not as a suggestion as to an order for the steps.

What is claimed is:

1. A head-mounted spectrum sensing device, comprising: an acoustic system, a magnetic system, an ultrasound system, and a control and processing system; wherein:
the acoustic system is configured to obtain blood velocity and a first thermal image, and mark the blood velocity at a corresponding position in the first thermal image to generate a thermal image with blood velocity information;
the magnetic system is configured to obtain magnetic flux and a second thermal image, and mark the magnetic flux at a corresponding position in the second thermal image to generate a thermal image with magnetic flux information;
the ultrasound system is configured to obtain ultrasound echoes and a third thermal image, and then mark the ultrasound echoes at a corresponding position in the second thermal image to generate a thermal image with ultrasound information; and
the control and processing system is configured to receive and integrate the thermal image with blood velocity information, the thermal image with magnetic flux information, and the thermal image with ultrasound information to obtain a spectrum fusion information, and judge a risk of dementia according to the spectrum fusion information.

2. The head-mounted spectrum sensing device of claim 1, wherein the acoustic system comprising:
a surface acoustic sensing unit, configured to obtain blood velocity;
a first thermal sensing unit, configured to obtain temperature distribution of a brain and generate a first thermal image according the temperature distribution; and
a first fusion unit, configured to mark the blood velocity at a corresponding position in the first thermal image.

3. The head-mounted spectrum sensing device of claim 2, wherein the surface acoustic sensing unit is configured to predict whether there is a vascular occlusion according to the blood velocity.

4. The head-mounted spectrum sensing device of claim 2, wherein the surface acoustic sensing unit comprises a plurality of flexible surface acoustic wave sensors.

5. The head-mounted spectrum sensing device of claim 4, wherein the flexible surface acoustic wave sensor comprises a polyimide substrate and a zinc oxide piezoelectric film on the polyimide substrate.

6. The head-mounted spectrum sensing device of claim 2, wherein the acoustic system further comprises a first shell configured to support the surface acoustic sensing unit, the first thermal sensing unit, and the first fusion unit.

7. The head-mounted spectrum sensing device of claim 6, wherein the first shell has a helmet-like shape.

8. The head-mounted spectrum sensing device of claim 6, wherein the surface acoustic sensing unit is located inside of the first shell, and the first thermal sensing unit is located outside of the first shell.

9. The head-mounted spectrum sensing device of claim 1, wherein the magnetic system comprises:
a magnetic sensing unit, configured to obtain magnetic flux;
a second thermal sensing unit, configured to obtain temperature distribution of a brain and generate a second thermal image according the temperature distribution; and
a second fusion unit, configured to mark the magnetic flux at a corresponding position in the second thermal image.

10. The head-mounted spectrum sensing device of claim 9, wherein the magnetic sensing unit is configured to predict neurological changes of the brain.

11. The head-mounted spectrum sensing device of claim 9, wherein the magnetic sensing unit comprises a magnetic flux sensor based on Nitrogen-vacancy Diamond.

12. The head-mounted spectrum sensing device of claim 9, wherein the second thermal sensing unit is configured to predict whether there is inflammation in the brain according to the temperature distribution.

13. The head-mounted spectrum sensing device of claim 1, wherein the ultrasound system comprises:
an ultrasound sensing unit, configured to obtain ultrasound echoes;
a third thermal sensing unit, configured to obtain temperature distribution of the brain and generate a third thermal image according the temperature distribution; and
a third fusion unit, configured to mark the ultrasound echoes at a corresponding position in the third thermal image.

14. The head-mounted spectrum sensing device of claim 13, wherein the ultrasound sensing unit comprises an ultrasonic signal transmitting module and an ultrasonic signal receiving module.

15. The head-mounted spectrum sensing device of claim 14, wherein the ultrasound sensing unit is configured to transform the ultrasound echoes into an image via Fourier transform.

16. The head-mounted spectrum sensing device of claim 1, wherein the control and processing system comprises:
a fourth fusion unit, configured to analyze and integrate the thermal image with blood velocity information, the thermal image with magnetic flux information, and the thermal image with ultrasound information to obtain the spectrum fusion information; and
a judgment unit, configured to receive the spectrum fusion information and determine whether the user has a risk of dementia.

17. The head-mounted spectrum sensing device of claim 16, wherein the judgment unit is configured to compare the spectrum fusion information with a big data database to obtain a change value and judge a risk of dementia according to the change value.
